(19) United States
(12) Patent Application Publication
(10) Pub. No.: US 2026/0027188 A1
(43) Pub. Date: Jan. 29, 2026

(54) Method and Composition for Treating Conditions Associated with a Leaky Gut Barrier

(71) Applicant: Lonza Greenwood LLC, Greenwood, SC (US)

(72) Inventors: Vijaya Juturu, Greenwood, SC (US); Zainulabedin Saiyed, Greenwood, SC (US); Shane Durkee, Greenwood, SC (US)

(21) Appl. No.: 19/099,079

(22) PCT Filed: Jul. 28, 2023
(86) PCT No.: PCT/US2023/028932

§ 371 Date: Jan. 28, 2025

Related U.S. Application Data
(60) Provisional application No. 63/393,286, filed on Jul. 29, 2022.

(51) Int. Cl. A61K 38/39; A61K 35/747; A61P 1/00

(52) U.S. Cl. CPC A61K 38/39; A61K 35/747; A61P 1/00

(57) ABSTRACT

The present disclosure is directed to a method for treating a mammal with leaky gut syndrome or having pre-stages of leaky gut syndrome. The method includes administering a composition that includes a native or undenatured type II collagen and/or a probiotic.

### REFERENCE TO RELATED APPLICATIONS

This application is based upon and claims priority to and the benefit of U.S. Provisional Application No. 63/393,286, filed on Jul. 29, 2022, the contents of which are incorporated herein by reference in its entirety.

### BACKGROUND

In recent years, the use of collagen to treat various conditions has become exceedingly popular. Collagen is a protein that can be found in muscles, bones, skin, blood vessels, and in other parts of the body. There are various different types of collagen depending upon its function and form. For instance, Type I collagen, the most abundant collagen, is made of fibers found in tendons, ligaments, organs and skin. Type II collagen, on the other hand, primarily helps build cartilage, a major structural entity that sits on the surfaces of those bones which comprise articulating joints. Type III collagen is a major component of the extracellular matrix that makes up organs and skin. Type III collagen also forms blood vessels and tissue within the heart. Type IV collagen is found primarily in the skin as sheet-like structures in the cutaneous basal lamina.

The gastrointestinal (GI) tract is a series of hollow organs joined in a long, twisting tube from the mouth to the anus of an animal (e.g., a vertebrate animal such as a human). The hollow organs that make up the gastrointestinal (GI) tract are the mouth, the esophagus, the stomach, the small intestine, the large intestine (also known as the colon) which includes the rectum, and the anus. Food (both solid and liquid) enters the mouth and passes to the anus through the hollow organs of the GI tract.

Digestion works by moving food through the GI tract. Food enters the mouth and is broken down with chewing and the digestive juice saliva. It is then swallowed and moves through the esophagus into the stomach where stomach acid further breaks down the food. The digested food then passes into the small intestine, where it mixes with digestive juices, causing large molecules of food to break down into smaller molecules. The body then absorbs these smaller molecules through the walls of the small intestine into the bloodstream, which delivers them to the rest of the body. Waste products of digestion pass through the large intestine and out of the body through the anus as a solid matter called stool or feces.

Bacteria in the GI tract, also called gut flora or microbiome, help with digestion. Parts of the nervous and circulatory systems also play roles in the digestive process. Together, a combination of nerves, hormones, bacteria, blood, and the organs of the digestive system completes the complex task of digesting the food (e.g., solid and liquid) an animal consumes each day.

In a healthy gut, digested food is absorbed through a well-controlled mechanism of the cellular wall which forms an intestinal barrier. A leaky gut occurs when the intestinal barrier, or tight junctions and enterocytes (mucosal cells), in the gut do not function properly. The gut barrier acts as gatekeepers between the gut and the bloodstream. When these tight junctions loosen or the enterocytes are damaged, gastrointestinal issues start to occur and undigested food particles, infectious organisms, such as harmful bacteria, parasites, and yeast get into the bloodstream. The result is often inflammation, joint pain and allergies which can all lead to obesity and chronic diseases.

Leaky gut syndrome (also known as increased intestinal permeability) occurs when there are gaps in the intestinal walls, called tight junctions, loosen, creating a wide gap in the intestinal wall. These loosen gaps allow particles of undigested food, bacteria, toxins to pass to the bloodstream of the mammals through the loosen gaps in the intestinal tract. Leaky gut syndrome starts with a very small break in the intestinal lining or a loosen gaps which is not easily or quickly detected. By the time it is detected, the break in the GI tract may be substantial, and may lead to advanced symptoms in the mammal. It is also believe that leaky gut syndrome may be caused by intestinal inflammation.

Leaky gut syndrome has been linked to various ailments, including, but not limited to acme, eczema and other skin problems, autoimmune conditions, arthritis, fibromyalgia, food allergies, seasonal allergies, obesity, diabetes, depression and mood springs.

### SUMMARY

In one embodiment, the present disclosure relates to a method of treating a mammal with leaky gut syndrome or having pre-stages of leaky gut syndrome, said method comprising administering to the mammal an amount of a native or undenatured type II collagen composition in an amount sufficient to reduce the effects of leaky gut syndrome in the mammal. The native or undenatured type II collagen containing composition is administered in an amount from about 1 mg to about 1000 mg.

In one embodiment, the present disclosure relates to a method of treating a mammal with an inflamed intestine, said method comprising administering to the mammal an amount of a native or undenatured type II collagen composition in an amount sufficient to reduce the effects the inflamed intestine in the mammal. The native or undenatured type II collagen containing composition is administered in an amount from about 1 mg to about 1000 mg.

### DEFINITIONS

As used herein, the terms “about,” “approximately,” or “generally,” when used to modify a value, indicates that the value can be raised or lowered by 10% and remain within the disclosed aspect.

The term “therapeutically effective amount” as used herein, with, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “nutraceutical” and “ingredient” refers to any compound added to a dietary source (e.g., a fortified food or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved gut health can include without limitation canine, equine, feline, bovine, ovine, human, or porcine mammals.

As used herein, “healthy” refers to the absence of illness or injury.

As used herein, “probiotic cell mater” or “Probiotic” as used herein refers to one or more probiotic organisms, including viable bacterial and yeast cells, as well as paraprobiotics, which include the killed or inactivated cells of probiotic organisms and/or the crude cell fractions of probiotic organisms including probiotic derivatives, which include the processed cell components of probiotic organisms. However, as will be discussed in greater detail below, “percent recovery yield” of viable probiotics are based upon the amount of viable probiotic contained in the probiotic cell matter or probiotic prior to processing. Therefore, “percent recovery yield” is a percentage of the initial, pre-processing, viable probiotic recovered after processing or stability testing.

Other features and aspects of the present disclosure are discussed in greater detail below.

### BRIEF DESCRIPTION OF THE FIGURES

FIG. 1 depicts ulcerative colitis disease severity measured by disease activity index. Values are presented as percent change in different parameters versus the control group (DSS only).

FIG. 2 depicts histopathological scores. Values are presented as percent change in different parameters versus the control group (DSS only).

FIG. 3 depicts presence of IgA. Values are presented as percent change in different parameters versus the control group (DSS only).

FIG. 4 depicts presence of IgE. Values are presented as percent change in different parameters versus the control group (DSS only).

FIG. 5 depicts presence of FITC-DX4. Values are presented as percent change in different parameters versus the control group (DSS only).

FIG. 6 depicts presence of MCP-1. Values are presented as percent change in different parameters versus the control group (DSS only).

FIG. 7 depicts presence of TNF-α. Values are presented as percent change in different parameters versus the control group (DSS only).

FIG. 8 depicts presence of COX-2. Values are presented as percent change in different parameters versus the control group (DSS only).

FIG. 9 depicts presence of IL-6. Values are presented as percent change in different parameters versus the control group (DSS only).

FIG. 10 depicts presence of IL-1β. Values are presented as percent change in different parameters versus the control group (DSS only).

FIG. 11 depicts presence of ZO-1. Values are presented as percent change in different parameters versus the control group (DSS only).

FIG. 12 depicts presence of Occludin. Values are presented as percent change in different parameters versus the control group (DSS only).

FIG. 13 depicts presence of E-cadherin. Values are presented as percent change in different parameters versus the control group (DSS only).

FIG. 14 depicts presence of Claudin-3. Values are presented as percent change in different parameters versus the control group (DSS only).

FIG. 15 depicts presence of β-catenin. Values are presented as percent change in different parameters versus the control group (DSS only).

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to a method leaky gut syndrome or having pre-stages of leaky gut syndrome. The method includes administering to the mammal an amount of a native or undenatured type II collagen composition in an amount sufficient to reduce the effects of leaky gut syndrome in the mammal. Particularly, the present disclosure has found that administering a composition that includes a native or undenatured type II collagen, may unexpectedly improve the symptoms of leaky gut syndrome in a mammal. Furthermore, in one aspect, the composition of the present disclosure may also help to correct an imbalanced intestinal microbiome.

In one aspect, the composition including a native or undenatured type II collagen source may provide an anti-inflammatory effect, lessening the effect of leaky gut syndrome. The composition is administered as a nutritional supplement. Therefore, the composition of the present disclosure may provide many benefits to intestinal health, as well as greater benefits, than previously obtained with other treatments.

In general, the composition of the supplement of the present disclosure contains a type II collagen which is a native or undenatured type II collagen. Additionally other types of collagen may be used in combination with the native or undenatured type II collagen may include a mixture of native type II collagen and undenatured type II collagen, in addition to a further collagen, such as Type I, Type III. Type IV or Type II collagen which is denatured.

As indicated above, the composition contains a collagen, particularly a Type II collagen such as an undenatured Type II collagen. Type II collagen for use in the present disclosure can be obtained from any suitable source. For instance, the collagen can be derived from a variety of mammalian sources, avian sources, or can be obtained from various fish species or a combination thereof. For instance, the collagen can be obtained from salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II® from Lonza Greenwood LLC. UC-II® is a natural ingredient that contains a glycosylated, undenatured Type II collagen. The collagen source can also comprise a hydrolyzed collagen. The collagen source can also comprise a pure protein or active peptide fragments. In one embodiment, the collagen source can be free of any bone or bone material. In other embodiments, the collagen source can be free of any transforming growth factors (TGFs), bone morphogenetic proteins (BMPs), or both. In still another embodiment, the collagen source comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the Type II collagen in the tissue and formed into doses containing therapeutically effective levels of undenatured Type II collagen, said levels being generally in the amount of at least about 0.1 mg preferably from about 2 mg to about 500 mg of animal tissue in a dose. Being a natural product some variation from sample to sample is to be expected. These variations can be minimized by blending after comminution. The blending can be aided by analytical techniques which allow the measurement of the amount of undenatured Type II collagen and other constituents.

In one aspect, the total Type II collagen and/or total collagen source is present in the composition in an amount from about 1 milligram to about 5000 milligrams. For instance, the total Type II collagen and/or total collagen source can be present in the composition in an amount greater than about 1 milligrams, such as in an amount greater than about 10 milligrams, such as in an amount greater than about 15 milligrams, such as in an amount greater than about 20 milligrams, such as in an amount greater than about 25 milligrams, such as in an amount greater than about 30 milligrams. The total amount of Type II collagen and/or total collagen source present in the composition is generally be less than about 1000 milligrams, such as less than about 900 milligrams, such as less than about 800 milligrams, such as less than about 700 milligrams, such as less than about 600 milligrams, such as less than about 500 milligrams, such as less than about 400 milligrams, such as less than about 300 milligrams, such as less than about 200 milligrams, such as less than about 100 milligrams, such as less than about 80 milligrams, such as less than about 70 milligrams, such as less than about 60 milligrams, or any ranges or values therebetween. Additionally or alternatively, the total type II collagen and/or total collagen source may be present in the composition in an amount of about 30% to about 90% by weight, such as about 35% to about 85%, such as about 40% to about 80%, such as about 45% to about 75%, such as about 50% to about 70%, such as about 55% to about 65%, or any ranges or values therebetween.

In one aspect, the undenatured type II collagen may form all, or substantially all, of the total type II collagen, and therefore, may be present in the above discussed amounts. However, in one aspect, the undenatured type II collagen may account for about 1% to about 95% of the total type II collagen and/or total collagen source, such as about 2.5% to about 75%, such as about 5% to about 50%, such as about 10% to about 40% of the total type II collagen, or any ranges or values therebetween. Therefore, in one aspect, the undenatured type II collagen may be present in the composition in an amount of 0.1 mg to about 100 mg, such as about 0.5 mg to about 75 mg, such as about 0.75 mg to about 50 mg, such as about 1 mg to about 30 mg, or any ranges or values therebetween.

Furthermore, in one aspect, the type II collagen and/or collagen source may further include a preservative salt, such as potassium chloride. Thus, in one aspect, the total amounts of type II collagen or total collagen source discussed above may include type II collagen and/or undenatured type II collagen, alone or in combination with a further collagen, a preservative salt, or combinations thereof. In such as aspect, the total type II collagen, including native and undenatured type II collagen, may account for about 1% to about 99% of the total collagen source, such as about 2.5% to about 90%, such as about 5% to about 80%, such as about 7.5% to about 70%, such as about 10% to about 60%, such as about 15% to about 50%, such as about 20% to about 35%, or any ranges or values therebetween. Thus, in one aspect, the total amount of type II collagen, including native and undenatured type II collagen in the composition may be from about 1 mg to about 1000 mg, such as about 2.5 mg to about 500 mg, such as about 5 mg to about 250 mg, such as about 7.5 mg to about 100 mg, such as about 10 mg to about 40 mg. or any ranges or values therebetween. Of course, in one aspect, no preservative salt is used.

Additionally or alternatively, as discussed above, the collagen source is combined with a collagen peptide. The term “collagen peptide” (CP) as used herein refers to a peptide derived from collagen, collagen peptides are short chains of amino acids typically extracted from native (full-length) collagen such as via enzymatic hydrolysis (also called enzymatic hydrolysation). The degree of hydrolysis usually has an impact on the average molecular weight of the final product. The term “collagen peptide” may be used interchangeably and synonymous with the terms “hydrolyzed collagen” or “collagen hydrolysate”.

In a one embodiment, the collagen peptide as part of the current invention is derived from collagen obtained from animal skin, cartilage, bone, and/or connective tissue. In another embodiment, the collagen peptide as part of the current invention is derived from porcine, bovine and/or fish collagen. The collagen as disclosed herein is preferably derived from collagen type I and/or collagen type II.

The collagen peptide as taught in the current invention may comprise collagen which is hydrolyzed or partially hydrolyzed, wherein the collagen may be derived from any species of animal. In addition or alternatively, an animal herein may refer to any animal capable of providing connective tissue, and the connective tissue is used to prepare collagen peptide.

In one embodiment, the collagen as taught herein is derived from a cow. In another embodiment, the collagen as taught herein is derived from a pig. In yet another embodiment, the collagen as taught herein is derived from a fish.

In various embodiments, the collagen as provided in the combination of the current invention is a mixture of collagen from different sources, such as collagen originating from multiple animal species, and/or collagen originating from different tissues. For example, the collagen as provided in the combination of the current invention may be a mixture of collagen chosen from fish collagen, porcine collagen, and bovine collagen. In addition or alternatively, the collagen may be a mixture of collagen chosen from skin-, cartilage-, bone-, and/or connective tissue-derived collagen. The connective tissue as taught herein refers to a group of tissues present between various tissues, organs and cells, including but not limited to being present in the corpus callosum, skin, spines (e.g. antler), protrusions (e.g. humps, lion's milk), horns, head, brain, neck, ear, eye, nose, tongue, lip, mouth, esophagus, trachea, limbs, feet, toes, palms, claws, bones, cartilage, bone marrow, joints, membranes, hind, ligaments, tendons, tendons interval, tendon, rib, diaphragm, muscle, skeletal muscle, smooth muscle, intestine, esophagus, trachea, Venetian, belly, blood vessels, bladder, stomach, aorta, heart, liver, kidney, chest, lung, spleen, pancreas, egg, sperm, testis, ovary, nerve, gallbladder, belly.

In a particular embodiment, the collagen as taught herein is derived from the skin and/or skin connective tissue. In another preferred embodiment, the collagen as taught herein is derived from the hind. In another preferred embodiment, the collagen as taught herein is derived from cartilage. In yet another preferred embodiment, the collagen as taught herein is derived from bone.

In various embodiments of the current invention, the collagen peptide may be produced by the enzymatic hydrolysis or partial enzymatic hydrolysis of collagen, wherein the enzyme used for this purpose may be one or more selected from the group consisting of serine protease, alkaline protease, neutral protease, flavour protease, complex protease, thiol protease, bromelain, metalloprotease, aspartame, protease, carboxypeptidase, pepsin, chymotrypsin, trypsin, cathepsin K, chymotrypsin, papain, and subtilisin combination.

In a one embodiment of the present disclosure, the collagen peptide has a mean molecular weight (Mw) between 300 Da and 10000 Da, e.g. between 500 Da and 7500 Da, between 1000 Da and 6000 Da, between 1500 Da and 5000 Da, or between 1500 Da and 3000 Da. The molecular weight as disclosed herein is generally the weight averaged molecular weight Mw. In a more embodiment of the current invention, the collagen peptide has a mean molecular weight between 1500 Da and 3000 Da or a mean molecular weight between 3000 Da and 6000 Da.

The collagen peptide as taught herein may have a protein content of 75-99 wt. %, preferably 80-99 wt. %, more preferably 85-99 wt. %, most preferably 90-99 wt. %. In an embodiment, the collagen peptide as part of the combination as taught herein comprises glycine in an amount of 15-25 wt. %, proline in an amount of 9-13 wt. %, and hydroxy proline in an amount of 9-13 wt. %, all per total weight of the amino acids in the collagen peptide.

In an embodiment, the collagen peptide as part of the combination as taught herein comprises glycine in an amount of 5-50 wt. %, preferably in an amount of 10-30 wt. %, more preferably in an amount of 12.5-27.5 wt. %, even more preferably in an amount of 15-25 wt. %, most preferably in an amount of 17.5-22.5wt. %, all per total weight of the amino acids in the collagen peptide.

In a particular embodiment, the collagen peptide as part of the combination as taught herein comprises proline in an amount of 2-20 wt. %, preferably in an amount of 7-15 wt. %, more preferably in an amount of 8-14 wt. %, even more preferably in an amount of 9-13 wt %, most preferably in an amount of 10-12 wt. %, all per total weight of the amino acids in the collagen peptide.

In another embodiment, the collagen peptide as part of the combination as taught herein comprises hydroxyproline in an amount of 2-20 wt. %. preferably in an amount of 7-15 wt. %, more preferably in an amount of 8-14 wt. %, even more preferably in an amount of 9-13 wt. %, most preferably in an amount of 10-12 wt. %, all per total weight of the amino acids in the collagen peptide.

In a particular embodiment, the collagen peptide as part of the combination as taught herein has a molecular weight of 1750-2250 Da and comprises glycine in an amount of 17.5-22.5 wt. %, proline in an amount of 10-12 wt. %, and hydroxyproline in an amount of 10-12 wt. %, all per total weight of the amino acids in the collagen peptide.

In another embodiment, the collagen peptide as part of the combination as taught herein has a molecular weight of 4750-5250 Da and comprises glycine in an amount of 17.5-22.5 wt. %, proline in an amount of 10-12 g wt. %, and hydroxyproline in an amount of 10-12 g wt %, all per total weight of the amino acids in the collagen peptide.

In another exemplary embodiment, the collagen peptide as part of the combination as taught herein has a molecular weight of between 1500-3000 Da and comprises glycine in an amount of 17.5-22.5 wt. %, proline in an amount of 10-12 wt. %, and hydroxyproline in an amount of 10-12 wt. %, all per total weight of the amino acids in the collagen peptide.

In an embodiment, the collagen peptide is present in the combination in an amount of between 1 mg and 10,000 mg (10g), typically between 10 mg and 5,000 mg (5 g), more typically between 50 mg and 2500 mg, most typically 100 mg and 1000 mg, wherein the amount is the dry weight of the composition.

In addition to the collagen source and the collagen peptides, the composition may contain other beneficial components. For instance, the composition may contain one or more vitamins, minerals, essential fatty acids, amino acids, polyphenols stilbenoids, curcuminoids, tannins, flavones, flavanols, flavan-3-ols, flavanones, anthocyanidins, anthocyanins, isoflavones, flavanonols, proanthocyanidins, dihydroxybenzoic acids, carotenoids, and pyridine alkaloids. In one aspect, the composition may contain an effective amount of an antioxidant, an amino acid, an essential fatty acid, a polyphenol, or combinations thereof. In a further embodiment of the present disclosure, there may be a probiotic material combined with the collagen or used in place with the collagen ingredient.

For example, in one aspect the composition contains at least one vitamin, such as at least one of vitamin B. vitamin C, and vitamin E. Vitamins may be contained in the composition in an amount of from about 50 μg/g of supplement to about 5000 μg/g, such as about 100 μg/g to about 4500, such as about 250 μg/g to about 4000 μg/g, such as about 400 μg/g to about 3500 μg/g, or any ranges or values therebetween. The above ranges may be for any one vitamin alone or a total amount of all vitamins. In one aspect, vitamin E is present in the composition in an amount of about 100 μg/g to about 1000 μg/g, such as about 250 μg/g to about 750 μg/g, such as about 400 μg/g to about 600 μg/g, or any ranges or values therebetween. In another aspect, vitamin C is present in the composition in an amount of about 1000 μg/g to about 5000 μg/g, such as about 2000 μg/g to about 4000 μg/g, such as about 3000 μg/g to about 3750 μg/g, or any ranges or values therebetween.

Furthermore, in an aspect, the composition contains at least one mineral, such as at least one of potassium magnesium, zinc, or calcium. Minerals may be contained in the composition in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the composition contains potassium in an amount of about 9.5 mg/g to about 12 mg/g, such as about 9.75 mg/g to about 11.5 mg/g, such as about 10 mg/g to about 11 mg/g. or any ranges or values therebetween. Similarly, in one aspect, the composition contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the composition contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

The probiotic material in one aspect, for instance, the probiotic organism used in accordance with the present disclosure comprises a type of bacteria. The bacteria may be selected from various different phyla including strains selected from the Firmicutes, the Gracilicutes, or the Mendocutes, and would include Bacteroidetes, Actinobacteria, Proteobacteria, Lactobacteria, the Bacilli, Verrucomicrobia, Faecalibacteria, Thermophiles, and the Clostridias. Specific examples of probiotics that may be used include, but are not limited to, Escherichia coli, Bacteroides fragilis, various Bifidobacteria and Lactobacteria, including Lactobacillus casei, Bifidobacterium longum ssp. infantis, Lactobacillus johnsonii, Lactobacillus rhamnosus, Lactobacillus reuteri, Lactobacillus acidophilus, Lactobacillus paracasei, Lactobacillus plantarum, and Lactobacillus lundensis, Bacillus coagulans, Bacillus subtilis, Faecalibacteria prausnitzil, Enterococcus faecium, Streptococcus salivarius, Clostridia butyricum, Akkermansia muciniphila, or mixtures thereof.

In an alternative aspect, eukaryotic cells may be used as the probiotic organism. For instance, the probiotic organism may comprise Saccharomyces cerevisiae.

In another aspect, the probiotic organism can include bacteria or yeast cells that have been treated or altered but remain viable. For instance, probiotic organism cells that have been lyophilized, or freeze-dried, but can be reconstituted, or probiotic organism cells that have undergone heat treatment, but retain viability, may be used in accordance with the present disclosure.

Nonetheless, in one aspect, at least one probiotic is a Lactobacilli, a Bifidobacteria, or a combination thereof, or other probiotics that provide a benefit to a small intestine or a colon of a mammal. One particular probiotic material usable in the present disclosure is a Lactobacillus plantarum a probiotic strain isolated from Taiwanese pickled cabbage, which is sold by Lonza Greenwood, LLC as TWK10® probiotic. This particular probiotic maybe isolated by the process described in U.S. Pat. No. 10,188,685 entitled “Use of Lactobacillus plantarum composition for manufacturing anti-fatigue probiotic composition to improve exercise performance”, which is hereby incorporated by reference.

Paraprobiotics that may be used in accordance with the present disclosure include the killed or inactivated cells of probiotic organisms and the cell fractions of probiotic organisms. However, as discussed above, it should be understood that at least a portion of the probiotics incorporated into the lipid multiparticulate are viable probiotics. Thus, in one aspect, paraprobiotics can be used in conjunction with viable probiotics, and, in one aspect, the probiotic may not contain substantially any paraprobiotics/inactivated cells prior to incorporation into the lipid matrix. Nonetheless, in one aspect, the paraprobiotic may comprise bacteria or yeast cells that have undergone heat treatment and are no longer viable but are still able to provide health benefits when consumed or administered to a mammal. Alternatively, the paraprobiotic may comprise viable or nonviable cells possibly subjected to changes in pH, increased pressure, milling, or the like, and in various combinations.

In yet another aspect, the paraprobiotic may comprise crude cell fractions containing, for instance, metabolites or hydrolysates. For example, protein hydrolysates extracted from yeast may be used in accordance with the present disclosure.

Other paraprobiotics that may be used in accordance with the present disclosure include probiotic derivatives that may include processed cell components of probiotic organisms, mixtures of processed probiotic organism cell components, and mixtures of nutrients and one or more components derived from probiotic organism cells. In one embodiment, the probiotic derivative may comprise processed cell fractions which contain, for instance, metabolites or hydrolysates. In another aspect, a mixture made up of beneficial nutrients and metabolites that are produced by yeast, such as the EpiCor® brand fermentate, which is commercially available from Embria Health Sciences, LLC, may be used in accordance with the present disclosure.

The probiotic material may alternative be used alone without the undenatured collagen ingredient or may be used in combination with the undenatured collagen ingredient. Generally, the probiotic is typically administered to the mammal in an amount from about 0.5 Billion (B) Colony Forming units (CFU) to about 150 B CFU, for example, 1 B CFU to about 75 B CFU and more particularly between about 2.5 B CFU to 50 B CFU and most particularly between 10 B CFU to about 30 B CFU.

In one aspect, the composition is administered once per day, twice per day, three times per day, up to about five times per day. However, in one aspect, the composition is administered once per day.

Notwithstanding the final amounts of collagen, collagen peptide, and other additives contained in the composition, or delivery method used in one aspect, a mammal being administered the composition may exhibit an improvement in the side effects of leaky gut syndrome.

While various aspects and benefits have been discussed, in one aspect, the composition is incorporated into a suitable delivery form prior to incorporation into a dosage form as discussed below. In one aspect, the composition may be coated onto a seed in order to form a multi-particulate dosage form that may be used in one or more of the administrations discussed in greater detail below. For instance, these multi-particulates may contain a starter particle or pellet, also known as seeds, beads, nonpareils, micro-granules, or starter cores. The cores are formed of inert material such as sucrose or microcrystalline cellulose. These cores are used as starter material and layered with active ingredient compounds before being incorporated into a final oral dosage formulation.

In some embodiments, the one or more particles provided herein may be formulated into any suitable dosage formulation. For example, in certain aspects, the one or more particles provided herein may be placed into a capsule for delivery by oral ingestion. Exemplary capsules include hard gelatin capsules, soft gelatin capsules, HPMC capsules, as well as capsules made from other materials. The one or more particles may be suspended in an aqueous-based matrix or an oil-based matrix within the capsule itself. In certain embodiments where the particles are suspended in an aqueous-based matrix or an oil-based matrix, the aqueous-based matrix or oil-based matrix may additional include one or more active ingredients. In certain embodiments, the one or more particles may be contained within a monolithic enteric capsule suitable for providing a modified release profile when ingested.

Capsules normally include a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenans) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. See, e.g., U.S. Pat. Nos. 5,264,223, 5,756,123, and 5,756,123. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. See, e.g., EP 0401832, U.S. Pat. Nos. 3,493,407, 4,001,211, and 3,617,588, GB 1310697, and WO 2008/050209. The aforementioned manufacturing processes involve the use of solutions of the different ingredients that are needed for the making the telescoping fit hard capsule shells.

Hard capsules may be filled with active ingredients, such as the composition described herein, via procedures known in the art. Typically, active ingredients (e.g collagen peptide and type II collagen) are combined with various compatible excipients for ease of fill. The resulting fill may be a dry powder, a granulation, particles, lipid particles, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets. For example, in some embodiments, provided is a pharmaceutical composition that contains a capsule filled with the one or more particles disclosed herein. In some embodiments, the one or more particles have not been enterically coated for modified release or gastric protection.

Additionally or alternatively, in one aspect, the composition of the present disclosure may be included as an oil-in-water emulsion as a delivery form. Particularly, in one aspect, such an arrangement may allow one or more oil-soluble and/or one or more water-soluble active ingredients to be contained in the same delivery form. Alternatively, only oil-soluble components may be used (e.g. the collagen peptide and the Type II collagen), and the emulsion may be used to incorporate the composition into a water-based application.

Nonetheless, the oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible. In some embodiments, the gum arabic may be comprised of a 100% modified gum arabic, such as Ticamulsion® A-2010 gum arabic powder. In certain embodiments, the gum arabic may be a mixture or blend of gum arabic and modified gum arabic. For example, in certain embodiments, the gum arabic may comprise Ticamulsion® 3020.

In certain aspects, the oil-in-water emulsion contains from about 10% to about 30% by weight of gum arabic. In some embodiments, the oil-in-water emulsion contains from about 15% to about 25% by weight of gum arabic. In some embodiments, the oil-in-water emulsion contains less than about 20% by weight of gum arabic, such as less than 15%, such as less than 10%, such at less than 5%.

The oil-in-water emulsion may also contain water. In certain aspects, the oil-in-water emulsion contains deionized water. Still, in certain aspects, the oil-in-water emulsion may contain any water suitable for human ingestion and incorporation into dietary supplements designed for human ingestion.

The amount of water incorporated into the oil-in-water emulsion can vary depending on the desired hygroscopic and water-soluble ingredients that are incorporated into the oil-in-water emulsion. In certain aspects, the oil-in-water emulsion may contain from about 5% to 35% by weight of water. In some embodiments, the oil-in-water emulsion may contain from about 10% to about 30% by weight of water. In some embodiments, the oil-in-water emulsion may contain from about 15% to about 20% by weight of water. In some embodiments, the oil-in-water emulsion may contain less than about 20% by weight of water, such as less than about 15% by weight of water, such as less than about 10% by weight of water.

In certain aspects, the oil-in-water emulsions may have a water activity of less than about 0.6, such as less than about 0.55, such as less than about 0.5, such as less than about 0.45, such as less than about 0.40, such as less than about 0.35, such as less than about 0.30, such as less than about 0.25, such as less than about 0.20, such as less than about 0.15, such as less than about 0.10. Generally, water activity (aw) described the equilibrium amount of water available for hydration of materials. Water activity generally measures the partial vapor pressure of water in the solution and divides that value by the partial vapor pressure of water at the same temperature to give a final reading. Water activity (aw) can be measured according to the following:

$$a w = p / {p}^{⋆}$$

For instance, in one aspect, emulsions having water activities over 0.6 may lead to physical instability of the emulsions. Furthermore, it was discovered herein that emulsions having a water activity greater than 0.6 may cause disintegration or dissolve certain HPMC capsules.

In some aspects, the oil-in-water emulsion may contain one or more stabilizers or suspension promoting agents. For example, in certain aspects, the oil-in-water emulsion may contain one or more gum, such as gellan gum or xanthan gum. If included, the gellan gum or xanthan gum may be present in an amount of less than about 3.5% by weight of the oil-in-water emulsion, such as less than about 2.5% by weight, such as less than about 1.5% by weight, such as less than about 1.0% by weight, such as less than about 1.0% by weight.

In other aspects, the oil-in-water emulsion may contain one or more stabilizers such as silica. If included, silica may be present in an amount of less than about 2% by weight, such as less than about 1.5% by weight, such as less than about 1% by weight, such as less than about 0.5% by weight.

Furthermore, in one aspect, the oil-in-water emulsion may also contain one or more fat-soluble ingredients or nutrients. In certain aspects, the one or more fat-soluble ingredients or nutrients may be incorporated into the oil phase of the oil-in-water phase emulsion. Suitable fat-soluble ingredients include, but are not limited to retinol, vitamin E sourced from mixed tocopherols, beta carotene, ubiquinone, lecithin, sunflower lecithin, vitamin D, cannabinoids, hemp extracts, vitamin K. phosphatidyl choline, and combinations thereof.

In certain aspects, at least one or more fat-soluble ingredients may be incorporated in the oil-in-water emulsion in an amount of from about 0% by weight to about 50% by weight. For example, in some aspects, the oil-in-water emulsion contains less than about 50% by weight of one or more fat-soluble ingredients, such as less than about 40% by weight, such as less than about 30% by weight, such as less than about 20% by weight, such as less than about 10% by weight, such a less than about 5% by weight.

Moreover, in one aspect, the oil-in water emulsion may contain one or more additional antioxidants, in one or more of the water soluble phase, or the oil/fat soluble phase.

In some aspects, the oil-in-water emulsion disclosed herein may be provided in any suitable dosage form, such as capsules, tablets, gummy chewables, edible films, lozenges, powders, liquid suspensions, syrups, lipid micelles, spray-dried dispersions, nanoparticles, and the like. In one aspect, the dosage form is an oral dosage form such as a capsule. Capsules are well-known dosage forms that normally consist of a shell filled with one or more specific substances, i.e. the oil-in-water emulsion disclosed herein. The capsule shell may be a soft shell or a hard shell containing film-forming polymers, such as gelatin, modified starches, modified cellulosed, etc.

Furthermore, in an aspect, the oil-in-water emulsion may be formulated to be provided in a capsule. In certain aspects, the capsule may include any suitable HPMC-based capsule. Advantageously, the oral dosage form provided herein may include an HPMC-based capsule containing the oil-in-water emulsion disclosed herein. In one aspect, the HPMC-base capsule may include capsules manufactured by Lonza Greenwood LLC under the tradename of VcapsQ, Vcaps® Plus, or DRcaps®. In some aspects, the capsule may include an modified HPMC-capsule that contains gellan gum and/or phthalate. In certain embodiments, the capsule may include an HPMC capsule that has been treated so as to be acid resistant.

In one aspect, the capsule may contain a certain fill weight range or fill volume range. For example, in certain aspects, the capsule may be filled with from about 150 mg to about 1050 mg of the oil-in-water emulsion. In one aspect, the capsule may be filled with about 150 mg of the oil-in-water emulsion. Furthermore, in an aspect, the capsule may be filled with about 20 mg of the oil-in-water emulsion. Moreover, in an aspect, the capsule may be filled with about 280 mg of the oil-in-water emulsion. In some aspects, the capsule may be filled with about 380 mg o more of the oil-in-water emulsion, such as about 500 mg or more of the oil-in-water emulsion, such as about 700 mg or more of the oil-in-water emulsion, such as about 1,050 mg or more of the oil-in-water emulsion.

Alternatively, the oil-in-water emulsion may be contained in a nutritional product, such as a food product or in a beverage. For example, in certain aspects, the oil-in-water emulsion may be incorporated into a liquid nutritional product, such as a nutritional supplement or infant formula, to be consumed by a mammal. Furthermore, the oil-in-water emulsions provided herein may be added to any liquid nutritional product designed to provide nutritional supplementation to a mammal.

Nonetheless, in one aspect, the composition, either alone or as contained in one of the delivery methods above can be administered orally as a solid, liquid, suspension, or gas. The composition may be administered via buccal or sublingual administration. In one embodiment, the composition may be administered as a capsule, tablet, caplet, pill, troche, drop, lozenge, powder, granule, syrup, tea, drink, thin film, seed, paste, herb, botanical, and the like.

Nonetheless, in addition to being administered orally, the composition can also be administered using other routes including intranasal, intravenous, intramuscular, intragastric, and the like.

When the composition, either alone or as part of a delivery form is combined with a food or beverage composition, the food or beverage composition may comprise any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, or the like.

The composition of the present disclosure may further comprise one or more excipients as further additives in the composition in conjunction with the collagen peptide and the collagen source. Exemplary but non-limiting excipients and/or additives include anti-adherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, and waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, licorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Ltd.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the pharmaceutical composition may comprise silica. For instance, the oxide particles may comprise precipitated silica particles. The silica particles may have a particle size (d50, laser diffraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m²/g, such as greater than about 130 m²/g, such as greater than about 150 m²/g, such as greater than about 170 m²/g, such as greater than about 200 m²/g, such as greater than about 220 m²/g. The specific surface area is generally less than about 500 m²/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Once the above components are combined together to form the composition, the composition can optionally be combined with a coating material. In one embodiment, for instance, the composition may comprise a granular composition to which a coating material is applied that contains a fat. The coating material, for instance, may comprise a hydrogenated oil, such as hydrogenated palm oil. In one particular embodiment, the coating material may comprise hydrogenated palm oil combined with palm stearin. In one embodiment, the hydrogenated oil may be present in the pharmaceutical composition in an amount from about 5% to about 35% by weight. The palm stearin, on the other hand, may be present in the pharmaceutical composition in an amount from about 2% to about 10% by weight. When present together, the weight ratio between the hydrogenated palm oil and the palm stearin may be from about 10:1 to about 1:1, such as from about 6:1 to about 2:1. In one embodiment, the hydrogenated palm oil and the palm stearin are present at a weight ratio of about 4:1.

Collagen is literally the “glue” that holds our body together, including muscles, bones, skin, blood vessels, tendons, and the lining of your digestive system. A healthy gut has a protective lining made up of protein cells which is mostly impermeable or sealed. The amino acids found in collagen help to rebuild this lining by providing the building blocks for a healthy gut. Collagen contains amino acids glycine, proline, and hydroxyproline that are needed to repair and rebuild the endothelial tissue in the digestive system

Administering undenatured type-II collagen to a mammal will reduce the intestinal inflammation, by normalizing the gut immune response. Undenatured type-II collagen is absorbed in the and initiates cell signaling that uses the body's natural repair mechanism to repair parts of the body of the mammal. Undenatured Type-II collagen is known travel to the intestine to the area called the Peyer's Patch in the gut, which is gut-associated lymphoid tissue (GALT). At the Peyer's Patch, the undenatured type-II collagen induces an immune response (e.g. T REGS) which is known to modulate inflammation. The T REG generated by the bodies response to the undenatured type-II collagen, will leave the gut through the lymphatic system and enter circulation in the blood stream. When the T reg cell arrive at body sites have inflammation, they will and rebuild the junctions of the produce anti-inflammatory cytokine (IL-10, TGF-β) that may help rebuild the tight junctions in the intestinal mucosal cells of the intestinal walls.

In a similar manner, the probiotic ingredient describe above may also reduce the intestinal inflammation, by normalizing the gut immune response, alone or in combination with the undenatured collagen ingredient.

Nonetheless, certain embodiments of the present disclosure may be better understood according to the following example, which is intended to be non-limiting and exemplary in nature.

### EXAMPLE 1

In vivo rat model testing was completed using Female Wistar and Sprague Dawley rats, aged 6 weeks. A positive control group was administered 3% Dextran sulfate sodium (DSS) to induce a clinically relevant loss of intestinal barrier function. A control group was not administered DSS nor undenatured type-II collagen. An investigational group was administered undenatured type-II collagen (UC-II® collagen from Lonza Greenwood LLC) in dose of 40 mg Human Equivalent Dose (HED) and 80 mg HED. The investigational group was also administered 3% DSS. The dosing for each group is shown in TABLE 1. All groups were fed a standard and received water. The positive control and the investigational group had 3% DSS in the water and the control did not. The study duration was two weeks.

TABLE 1

| Groups | Treatment A | Animal dose |
|---|---|---|
| Control | Control | — |
| DSS | 3% DSS | — |
| Dose 1 | 3% DSS + undenatured | 0.66 mg undenatured collagen/ |
| | collagen (dose 1) | kg/rat/day (40 mg HED) |
| Dose 2 | 3% DSS + undenatured | 1.32 mg undenatured collagen/ |
| | collagen (dose 2) | kg/rat/day (80 mg HED) |

After two weeks, each rat in the study was measured for: 1) Disease Activity Index, 2) Histopathological Score, 3) Immune Markers, 4) Intestinal Permeability, 5) Inflammatory Markers, and 6) Intestinal Tight Junction Proteins.

The results of each test method for all groups are graphically shown in the figures. The figures depict bar graphs comprising four bars, which represent each group, from left to right: control, DSS, dose 1, and dose 2. The results of each test method for the investigational groups administered 40 mg HED of undenatured type-II collagen and 80 mg undenatured type-II collagen are shown in TABLE 2 as a percent change in different parameters versus the positive control group (DSS only).

TABLE 2

| | Disease | | | | | |
|---|---|---|---|---|---|---|
| | Activity | | Immune | Intestinal | Inflammatory | Intestinal Tight |
| Test products | Index | Histology | Markers | Permeability | Markers | Junction Proteins |
| DSS + | | + | + | + | + | + |
| Undenatured | | (18% ↓)* | IgE (17% ↓)* | (12% ↓)* | MCP-1 (21% ↓)* | ZO1 (41% ↑)* |
| Type-II Collagen | | | IgA (19% ↓)* | | TNF-a & IL-6 | Occludin (56% ↑)* |
| dose 1 - (0.66 | | | | | (23%↓)* | Claudin 3 (59% ↑)* |
| mg/kg; 40 mg | | | | | IL-1b (18%↓)* | B-Catenin (85% ↑)* |
| HED | | | | | COX-2 (19% ↓)* | E Cadehin (46% ↑)* |
| Undenatured | | | | | | |
| Type-II Collagen) | | | | | | |
| DSS + | + | ++ | + | + | + | + |
| Undenatured | (24% ↓)* | (30% ↓)* | IgE (22% ↓)* | (17%↓)* | MCP-1 (26% ↓)* | Occludin (64% ↑)* |
| Type-II Collagen | | | IgA (24% ↓)* | | IL-1b (23% ↓)* | E Cadehin (60% ↑)* |
| dose 2 - (1.32 | | | | | ++ | ++ |
| mg/kg; 80 mg | | | | | TNF-a (28%↓)* | ZO1 (73% ↑)* |
| HED | | | | | IL-6 (29% ↓)* | Claudin-3 (103% ↑)* |
| Undenatured | | | | | COX-2 (25% ↓)* | B-catenin (117% ↑)* |
| Type-II Collagen) | | | | | | |

A description of the study methodology, outcome, and interpretation is shown in TABLE 3. In summary, the results indicated that all groups containing undenatured type-II collagen significantly inhibited DSS-induced Ulcerative Colitis effects compared to DSS alone in most measures.

TABLE 3

| | | What are the | What do they |
|---|---|---|---|
| Test Method | Outcomes | results | mean? |
| Disease | Percent weight | ↓ DAI | Reduced |
| Activity | loss, stool | | disease |
| Index | consistency, | | severity |
| | gross bleeding | | |
| Histology | Colon | Improved Colon | Reduced |
| | Inflammation, | Lining | disease |
| | Extent, | Parameters | severity |
| | Regeneration, | | |
| | Crypt Damage | | |
| | and Involvement | | |
| Immune | IgA, IgE, | ↓ IgA, IgE, | Reduced |
| markers | Endotoxin | Endotoxin | disease |
| | | | severity |
| Intestinal | FITC-DX4 | ↓ FITC-DX4 | Healthier |
| Permeability | | | intestinal |
| | | | lining |
| Inflammatory | MCP-1, TNF-α, | ↓ MCP-1, TNF-α, | Reduced |
| markers | IL-6, IL-1β, | IL-6, IL-1β, | inflammatory |
| | COX-2 | COX-2 | response |
| Intestinal | ZO-1, Occludin, | ↑ZO-1, Occludin, | Improved tight |
| Tight | Claudin-3, | Claudin-3, | junction |
| Junction | β-catenin, | β-catenin, | structure |
| Proteins | E-cadherin | E-cadherin | |

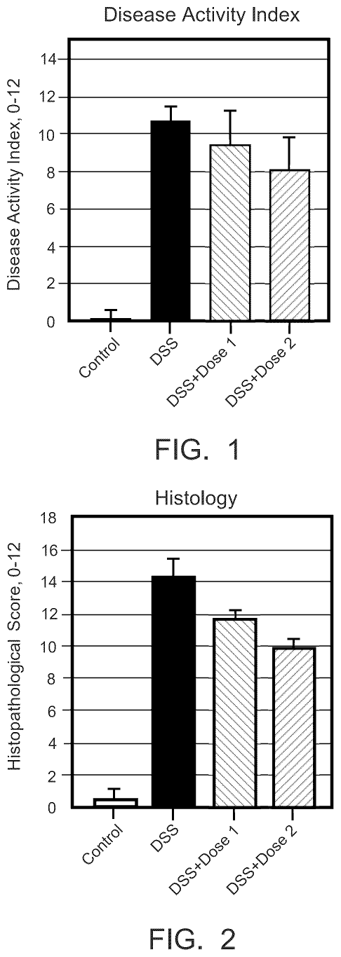

FIG. 1 depicts ulcerative colitis disease severity according to disease activity index data based on parameters observed in the control group that was administered only DSS. Measured parameters included percent weight loss, stool consistency, and gross bleeding. A reduction in the disease activity index scores was observed in groups that were administered 40 mg HED of undenatured type-II collagen and 80 mg undenatured type-II collagen, indicating decreased ulcerative colitis severity.

FIG. 2 depicts histopathological scores, which indicate disease severity in the colon lining by measuring the extent of colon inflammation and regeneration and crypt damage and involvement. Decreased histopathological scores were observed in groups that were administered 40 mg HED of undenatured type-II collagen and 80 mg undenatured type-II collagen, suggesting dose dependent and additive effects of the treatments on disease severity.

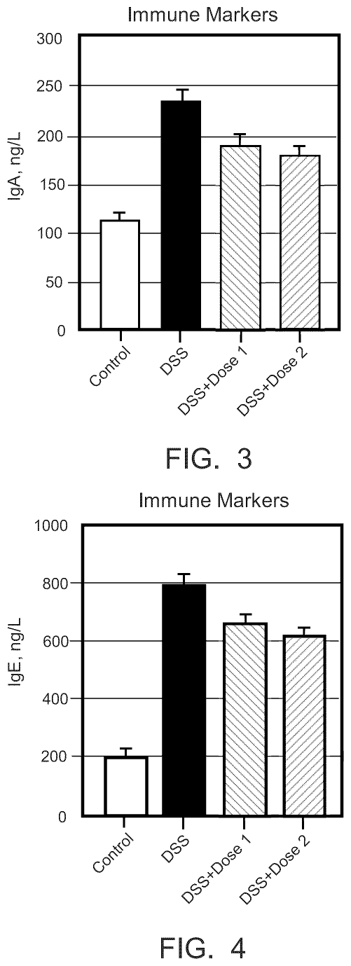

FIG. 3 and FIG. 4 depict presence of immune markers IgA and IgE, respectively. Reduced presence of immune markers was measured in groups that were administered 40 mg HED of undenatured type-II collagen and 80 mg undenatured type-II collagen, suggesting reduced disease severity.

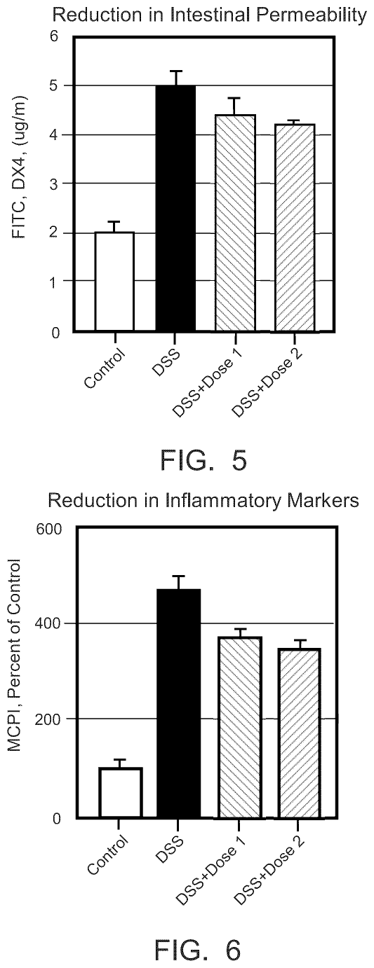

FIG. 5 depicts presence of FITC-DX4. Reduction of FITC-DX4 in groups that were administered 40 mg HED of undenatured type-II collagen and 80 mg undenatured type-II collagen suggests improved intestinal function and health.

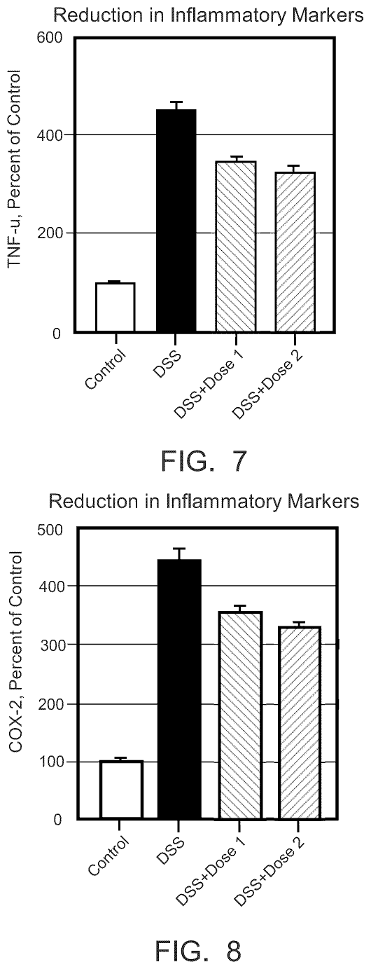

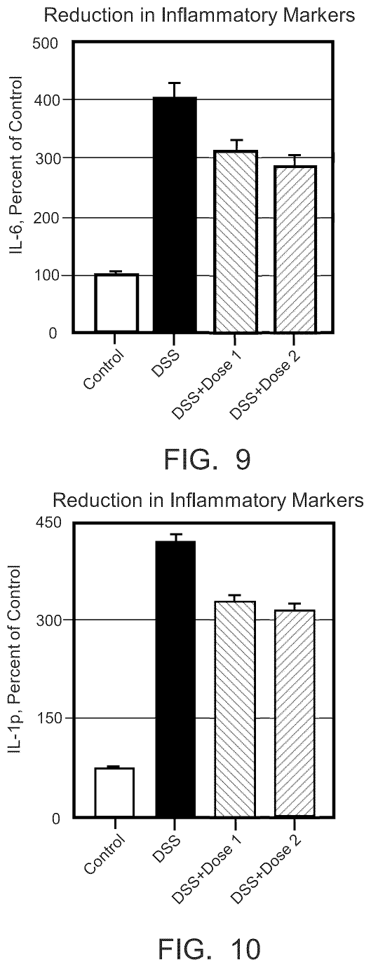

FIGS. 6-10 depict presence of inflammatory markers: MCP-1, TNF-α, COX-2, IL-6, and IL-1β. Reduced presence of inflammatory markers were observed in groups that were administered 40 mg HED of undenatured type-II collagen and 80 mg undenatured type-II collagen, suggesting reduction in disease induced inflammation. Reduced inflammatory marker presence was dose dependent for TNF-α, COX-2, and IL-6.

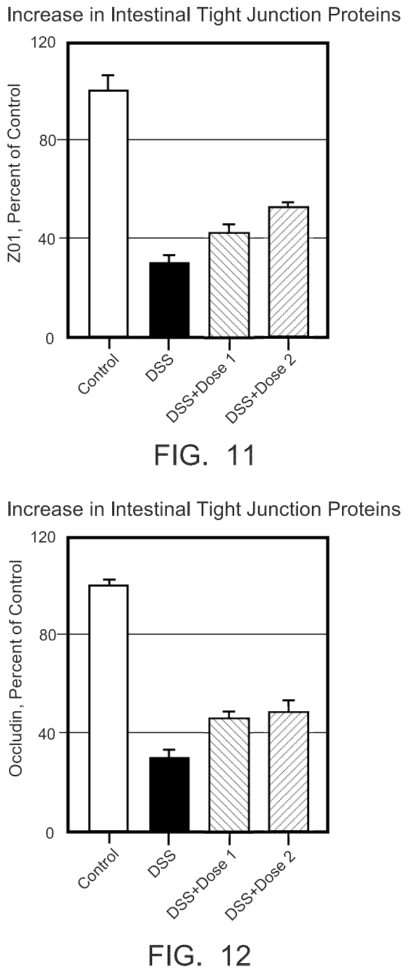

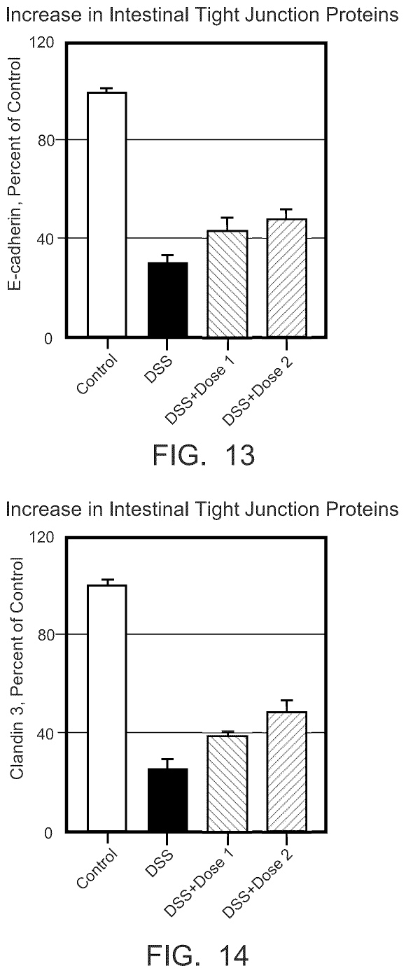

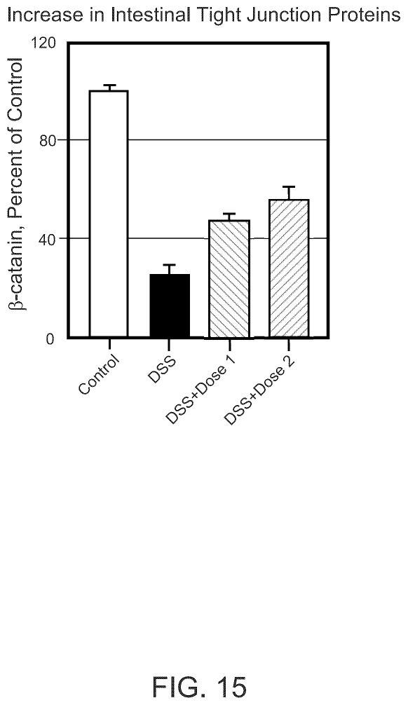

FIGS. 11-15 depict the presence of intestinal tight junction proteins: ZO-1, Occludin, E-cadherin, Claudin-3, and β-catenin. Increased presence of intestinal tight junction proteins was observed in groups that were administered 40 mg HED of undenatured type-II collagen and 80 mg undenatured type-II collagen, suggesting improved tight junction structure and performance and reduction in disease severity.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A method of treating a mammal with leaky gut syndrome or having pre-stages of leaky gut syndrome, said method comprising:
administering to the mammal an amount of a native or undenatured type II collagen composition in an amount sufficient to reduce the effects of leaky gut syndrome in the mammal,
wherein the native or undenatured type II collagen containing composition is administered in an amount from about 1 mg to about 1000 mg.

2. The method of claim 1, wherein the native or undenatured type II collagen composition is administered one to 4 times daily.

3. The method of claim 2, wherein the native or undenatured type II collagen composition is administered prior to a meal.

4. The method of claim 2, wherein the native or undenatured type II collagen composition is administered once per day.

5. The method according to claim. wherein the native or undenatured type II collagen composition is administered in an amount of about 5 mg to about 500 mg per dose.

6. The method of claim 1, wherein the native or undenatured type II collagen composition is administered to the mammal in an amount sufficient to reduce at least one immune marker, wherein the at least one immune marker comprises IgA, IgE, or Endotoxin.

7. The method of claim 1, wherein the native or undenatured type II collagen composition is administered to the mammal in an amount sufficient to reduce the presence of at least one inflammatory marker, wherein the at least one inflammatory marker comprises MCP-1, TNF-α, COX-2, IL-6, or IL-1β.

8. The method of claim 1, wherein the native or undenatured type II collagen composition is administered to the mammal in an amount sufficient to increase at least one intestinal tight junction protein, wherein the at least one intestinal tight junction protein comprises ZO-1, Occludin, E-cadherin, Claudin-3, or β-catenin.

9. The method according to claim 1, further comprising additionally administering a probiotic to the mammal.

10. The method according to claim 9, wherein the probiotic is L. plantarum.

11. The method according to claim 9, wherein the probiotic is administered in an amount of about 0.5 Billion (B) Colony Forming units (CFU) to about 150 B CFU.

12. A method of treating a mammal with an inflamed intestine, said method comprising:
administering to the mammal an amount of a native or undenatured type II collagen composition in an amount sufficient to reduce the effects of inflamed intestine in the mammal,
wherein the native or undenatured type II collagen containing composition is administered in an amount from about 1 mg to about 1000 mg.

13. The method of claim 12, wherein the native or undenatured type II collagen composition is administered one to 4 times daily.

14. The method of claim 13, wherein the native or undenatured type II collagen composition is administered prior to a meal.

15. The method of claim 14, wherein the native or undenatured type II collagen composition is administered once per day.

16. The method according to claim 12, wherein the native or undenatured type II collagen composition is administered in an amount of about 5 mg to about 500 mg per dose.

17. The method according to claim 12, further comprising additionally administering a probiotic to the mammal.

18. The method according to claim 17, wherein the probiotic comprises L. plantarum.

19. The method according to claim 17, wherein the probiotic is administered in an amount of about −0.5 Billion (B) Colony Forming units (CFU) to about 150 B CFU.

20. A method of treating a mammal with leaky gut syndrome or having pre-stages of leaky gut syndrome, said method comprising:
administering to the mammal an amount of a probiotic in an amount sufficient to reduce the effects of leaky gut syndrome in the mammal,
wherein the probiotic is administered in an amount from about 0.5 Billion (B) Colony Forming units (CFU) to about 150 B CFU.

21. The method according to claim 20, wherein the probiotic comprises L. plantarum.

22. A method of treating a mammal method of treating a mammal with an inflamed intestine, said method comprising:
administering to the mammal an amount of a probiotic in an amount sufficient to reduce the effects of inflamed intestine in the mammal,
wherein the probiotic is administered in an amount from about 0.5 Billion (B) Colony Forming units (CFU) to about 150 B CFU.

23. The method according to claim 22, wherein the probiotic comprises L. plantarum.
